(19) United States
(12) Patent Application Publication
(10) Pub. No.: US 2007/0248651 A1
(43) Pub. Date: Oct. 25, 2007

(54) Formulation for treatment of acne

(76) Inventors: Ellie Sawits, New York, NY (US); Peter C. Dea, San Francisco, CA (US); Laura Tringale, Fremont, CA (US)

Correspondence Address: Thomas M. Freiburger, P.O. Box 1026, Tiburon, CA, 94920, US

(21) Appl. No.: 11/483,234

(22) Filed: Jul. 7, 2006

Related U.S. Application Data
(60) Provisional application No. 60/703,758, filed on Jul. 28, 2005.

(51) Int. Cl. A61K 36/53; A61K 9/68; A61K 31/714; A61K 31/525; A61K 31/51; A61K 31/385; A61K 31/355; A61K 31/195; A61K 31/045

(52) U.S. Cl. 424/440; 424/642; 514/52; 514/251; 514/276; 514/350; 514/356; 514/458; 514/474; 514/561; 514/642; 514/393; 514/729; 424/745; 424/774; 424/702; 514/440

(57) ABSTRACT

A vitamin, mineral, herbal, and micronutrient treatment for acne via support of the physiologic processes in the body which cause acne is disclosed. It is a targeted formula, designed specifically to address the causes of acne. This specific, unique formulation preferably in a gummy substrate for ease of administration, to induce a willingness on the part of the target population to take it, and to increase the bioavailability of the active ingredients. Other types of confection or edible vehicle can be used.

This application claims benefit from provisional application Ser. No. 60/703,758 filed Jul. 28, 2005.

### BACKGROUND AND SUMMARY OF THE INVENTION

The treatment of acne in the United States has focused largely on the external administration of creams, astringents and the like, with little success. The current invention results from the theory, formulated by the principal inventor, that acne is an internal, periodic condition resulting from a temporary physiologic disequilibrium in the human body and should therefore be treated as such. Acne is hypothesized then to be not a disease, but rather a periodic condition resulting from one or another changes in the body when the body needs additional nutritional support. The inventor has identified those changes believed to cause acne and combined sufficient vitamins and minerals in the formulation to address each of these causes. The changes may be stress, hormonal imbalance or the dietary inadequacies in the population.

Generally this invention encompasses the administration of condition-specific formulations of vitamins, minerals, herbs, micronutrients in a gummy candy (or other confection) substrate, where the combination of vitamins, minerals herbs and micronutrients are specifically designed and formulated in a unique combination, to support the human body's functions with regard to, and to strengthen its resistance to, stated conditions, in this instance to treat acne and for other formulations applied to other specific conditions.

Multi-vitamin pills and tablets, which are available in great quantities, are typically all-encompassing combinations which are deemed to be necessary for general well-being. Multi-vitamin products do not contemplate the treatment or alleviation of any particular condition. This invention refers to specifically designed combinations of vitamins, minerals, herbs and micronutrients to address particular needs pertaining to particular conditions of the human body. These targeted nutrients assume, as a foundation, a general level of health, which the multi-vitamins address and are in addition to those, to treat acne specifically.

Single vitamin products such as Vitamin C water or single component pills rely on the consumer to select those requirements he or she needs, and to administer them correctly. This invention provides the pre-selected components for a particular condition and frees the consumer from having to study the scientific literature to make a correct selection for a given condition. For example, the administration of calcium supplements to women, a very popular product, ignores the consequent magnesium and phosphorus relationships to calcium, and may result in a either a magnesium or phosphorus deficiency. Thus self-administration without knowledge is potentially a problem.

This invention does not cover either the administration of a single vitamin or mineral, or the scattershot administration of multiple vitamins and minerals thrown at a problem with the hope that some of the vitamins will hit their target.

Pursuant to the invention, a preferred embodiment of gelatin based gummy candies are used as a delivery system of a specific blend of vitamins, minerals and herbs for the treatment of acne. The gummy candy is formulated with sugar alcohols to produce a sugar free product, and fruit flavored to improve palatability and acceptability of the finished product.

The uniqueness of the vitamin and mineral blend is the basis for this invention. Additionally, the absence of sugar in the product enhances the effectiveness of the vitamin blend.

The use of a gummy candy to deliver a micronutrient is not new technology. However, the use of the system to deliver a blend specific for the treatment of acne is unique with this product.

The patent and scientific literature is full of nutritional and vitamin supplement formulations, but there is no disclosure suggesting the present invention for protection and treatment of acne. Even though some of this invention's ingredients are available in generic multi-vitamin supplements, they are not provided in the combination or in the quantities necessary to treat acne specifically, as disclosed herein.

The formula is targeted to address the specific internal causes of acne, particularly in adolescents, the target cohort of 12 to 24 year olds.

The delivery vehicle of the formulation for acne is edible and may be, as noted above, a sugar-free gummy candy, but may also be in another candy, snack food form, or even a pill. The purpose of this invention is to make generally available the benefits of a targeted group of vitamins, minerals and micronutrients to the population without its having to select individual components to address health issues, but rather to have pre-selected elements incorporated in a single vehicle and for a single purpose. A further object is to provide ease of administration via the gummy candy form, encourage usage of such scientifically selected vitamins, minerals, herbs and nutrients for the support of specific body functions, and eliminate the need to swallow pills, a traditional barrier to the administration of such substances, especially for the target cohort of teens and young adults.

We have tested the formulation in over 200 test candidates ranging in age from 16 to 54, by one of the inventors in about 80 persons and independently in 120 persons. The formulation has resulted in a better than 70% efficacy rate overall. The formulation was calculated to be the minimum amount of micronutrients necessary to have the desired effect. This was done to assure that the target population which has experience eating gummy candies, get the desired effect and still be able to eat many more than the recommended daily amount of two per day with no untoward effects. As a result the ranges specified in this application utilize a minimum in the formulation, as produced, but, in fact, may be much larger in actuality, with no negative effects.

The maximum ranges provided in the table under Brief Manufacturing Description are considered normal amounts of administration for each of the components in the formula, and well within the range such that there is virtually no possibility of overdose. The use of a minimum amount pertains more to the target population being ages 12 to 24 and the candy-like format, than to the possible amounts that can be safely administered to a human. Thus the maximum amounts of the formulation would be therapeutic amounts well within a reasonable established dosage level for each constituent component, and thus able to be included at such levels. Thus this application discloses a range of absolute dosage levels unrelated to the vehicle in which they are provided. The provision of the disclosed formula with the disclosed ranges in a pill, capsule, drink or any more conventional vitamin, mineral and micronutrient vehicle is thus also contemplated by the invention.

### Research

The regulation of the skin, as the largest excretory organ of the human body, and consequently, the body's ability to eliminate acne, is supported by vitamins, minerals, herbs and micronutrients (15) (67) (68). It is the contention of this application that acne is an internal phenomenon, which manifests itself externally, but has as its root causes, internal changes in the body. This application purports to address the causes of acne, rather than its effects.

Vitamin B1, pantothenic acid, and other B vitamins have all been shown to play a role in wound healing in animal studies. (6) (8) (14). The inventors believe that wounds have a correlative in acne, including inflammation, infection and tissue destruction. The B vitamins play a role in hormonal regulation (10) (12), as does Vitamin E. (13) (21) (44). It has often been observed that hormonal changes affect acne, both the periodic form of the menstrual cycle and the surges of adolescence. Any substance which assists in the control and regulation of hormones would thus, in the view of the inventors, have a positive effect on acne. The B vitamin complex has also been shown to support the immune system in general (28), and B5 and B6 particularly for acne support (36) (40) (60). The ability of the body to ward off infection clearly has a role in the prevention of acne.

Vitamin C is needed to support the human immune system (48). The role of bacteria in acne pertains to immunity levels. Vitamin C is also crucial in the making of collagen that strengthens skin, muscles, and blood vessels, in support against inflammation (57), and to ensure proper wound healing (68). Preliminary human studies suggest that vitamin C supplementation in non-deficient people can speed healing of various types of wounds and trauma, including surgery, minor injuries, and skin ulcers. In addition Vitamin C is instrumental in support of the immune system and stress-related difficulties. (7)(16) (17) Each of these has a correlation to acne and the conditions which leave the body vulnerable to it.

Zinc assists in the repair of wounds (3)(4) (41) (42) (43) (50), the amelioration of acne (22) (23) (35) (69) and herpes simplex (26) (27) (29), immunity and inflammation (54). Both exercise and sleep-deprivation are shown to diminish internal zinc (38), both common occurrences in the target cohort. Stress and lack of sleep are notorious states for teens. Zinc may also play a role in hormonal imbalance correction (51) (70).

Vitamin E has been shown to help in ulcerative lesions, general cell injury and in scarring of the skin (45) (46) (47). Vitamin E also provides support for collagen (61). Animal studies have shown that supplementing with Vitamin E can decrease the formation of unwanted adhesions following a surgical wound. Vitamin E may also play a role in improving the immune system (53) (71). Vitamin E plays a role in hormonal balance.

Sugar consumption has a negative effect on the skin (2) (19). Nicotinamide has been shown to assist in the glucose levels of humans (49) (52). Given the sugar-laden diets of the U.S. population, and teens in particular, glucose regulation is important in acne prevention.

Bioavailability utilizing a slurry of vitamins, minerals, herbs and micronutrients in a chewable form is increased over encapsulated equivalents (39) or when administered in a pill format.

Several gummy multi-vitamin products have been available in the market. They are largely designed for children 2 to 4 years old, and 4 years old and above. Examples are sold under the trademarks L'IL CRITTERS, MY FIRST FLINTSTONES, RHINO GUMMY BEAR VITAMINS, and YUMMI BEAR VITAMINS. These gummy vitamins are intended to provide vitamin and mineral supplements for general health. They are not designed for skin care and not for acne problems. Importantly, these gummy multi-vitamins have about 3 or 4 grams of sugar per dose, which directly counteracts at least the B vitamins and Vitamin C directly. When the component nutritional contents of these gummy multi-vitamins are analyzed, they are found to include some B vitamins, but not all. In particular, the B vitamins which are bitter-tasting are not included. We believe that they are excluded, not because they are not efficacious, but because the successful manufacture of a good-tasting product is too difficult to include them.

Existing gummy vitamins include Vitamins A, D, calcium and magnesium, to achieve general welfare, none of which are included in the present invention, which specifically looks to include only those elements which are needed for the control of acne.

### DESCRIPTION OF PREFERRED EMBODIMENTS

1. In particular, this disclosure covers a unique nutritional vitamin, mineral, herbal formulation for the therapeutic purpose of strengthening the body's defenses against the internal processes which cause acne. The formula preferably includes full-spectrum B vitamins, not a selection of the B's, since an excess of one B can result in a deficiency of another. The formula also includes Vitamin C for its anti-inflammatory, anti-viral and anti-oxidant characteristics. The formula also includes zinc for its skin-healing properties, and includes Vitamin E for its hormone regulation properties.

In particular invention encompasses formula ranges as cited below (under Brief Manufacturing Description).

The invention further encompasses all edible forms of each component, when combined with the others, e.g. zinc as zinc sulfate or zinc acetate.

It is well known that the vitamin B complex components interact with each other, so that a surfeit of one will diminish another (72,73,74,75,76). As a result it is desirable to include substantially the full range of B vitamins in the formula, so as not to cause such a depletion. This invention is believed to provide the minimum amount of each vitamin, mineral and micronutrient necessary to be an effective amount (see minimums in the table below), assuming two pills, tablets or candies are taken daily. The table below provides, also, what an approximate maximum amount of the formula would be if administered in a pill or capsule form (two per day). It is the combination of these elements which gives the resulting efficacy against the disequilibria which cause acne. In some cases components preferably are included to broaden the application of the formulation, since there are apparently five or more different causes of acne for different individuals. Certain vitamins and minerals are effective against certain acne causes, but inclusion of components to treat several of the causes makes the formulation effective for more individuals. Thus this invention encompasses the formula ranges as described, well within the normal administration levels of each component, separately.

2. Original use of an edible formulation in a gummy vitamin format for strengthening the body's defenses against acne.

3. Use of a gummy vitamin format targeted for a therapeutic purpose to a particular malady or condition.

4. Easy-to-use and pleasant-tasting delivery system, which encourages the administration of the vitamin, mineral and herbal therapeutic formulation to control acne.

5. The deliberate maintenance of a slight vitamin, mineral, herbal, micronutrients aftertaste to inform the palate that although the product is in a candy substrate, the product has a therapeutic purpose.

6. Packaging in a fun, multi-colored, designed container to attract the target cohort of ages 12 to 24. The presentation of the formula in a fun package, with a non-medicinal face, for greater acceptability of the product, in the target cohort of 12 to 24 year olds.

7. Enhanced bioavailability of vitamins, minerals, herbs and micronutrients via mastication of a chewable gummy substrate rather than relying on the gastro-intestinal tract's ability to break down the substrate, which it does inefficiently, to treat a particular condition.

8. The avoidance of sugar as a recipe component, which would have the effect of diminishing and neutralizing the therapeutic impact of several of the active ingredients, and so be counter-productive.

9. A preferred combination is Vitamin E, in any of its various forms in the range of about 13 to 100 i.u., biotin in the range of about 3 mcg to 100 mcg, folic acid in the range of about 13 mcg to 100 mcg, niacin in any of its forms in the range of about 3 mg to 100 mg, PABA from about 3 mg to 100 mg, pantothenic acid from about 3 mg to 100 mg, Vitamin B1 from about 3 mg to 100 mg, Vitamin B12 from about 3 mcg to 50 mcg, Vitamin B2 (riboflavin) from about 3 mg to 100 mg, Vitamin B6 (Pyridoxine) from about 3 mg to 100 mg, Vitamin C (ascorbic acid) in its various forms from about 25 mg to 1000 mg, zinc in its various bioavailable forms from about 1.67 mg to 75 mg, choline from about 3 mg to 100 mg, inositol from about 3.33 mg to 100 mg.

10. Another combination would be Vitamin E, in any of its various forms in the range of about 13 to 100 i.u., folic acid in the range of about 13 mcg to 100 mcg, niacin in any of its forms in the range of about 3 mg to 100 mg, PABA from about 3 mg to 100 mg, pantothenic acid from about 3 mg to 100 mg, Vitamin B1 from about 3 mg to 100 mg, Vitamin B12 from about 3 mcg to 50 mcg, Vitamin B2 (riboflavin) from about 3 mg to 100 mg, Vitamin B6 (Pyridoxine) from about 3 mg to 100 mg, Vitamin C (ascorbic acid) in its various forms from about 25 mg to 1000 mg, zinc in its various bioavailable forms from about 1.67 mg to 75 mg, choline from about 3 mg to 100 mg, inositol from about 3.33 mg to 100 mg.

11. Another combination would be Vitamin E, in any of its various forms in the range of about 13 to 100 i.u., biotin in the range of about 3 mcg to 100 mcg, niacin in any of its forms of in the range of about 3 mg to 100 mg, PABA from about 3 mg to 100 mg, pantothenic acid from about 3 mg to 100 mg, Vitamin B1 from about 3 mg to 100 mg, Vitamin B12 from about 3 mcg to 50 mcg, Vitamin B2 (riboflavin) from about 3 mg to 100 mg, Vitamin B6 (Pyridoxine) from about 3 mg to 100 mg, Vitamin C (ascorbic acid) in its various forms from about 25 mg to 1000 mg, zinc in its various bioavailable forms from about 1.67 mg to 75 mg, choline from about 3 mg to 100 mg, inositol from about 3.33 mg to 100 mg.

12. Another combination would be Vitamin E, in any of its various forms in the range of about 13 to 100 i.u., biotin in the range of about 3 mcg to 100 mcg, folic acid in the range of about 13 mcg to 100 mcg, PABA from about 3 mg to 100 mg, pantothenic acid from about 3 mg to 100 mg, Vitamin B1 from about 3 mg to 100 mg, Vitamin B12 from about 3 mcg to 50 mcg, Vitamin B2 (riboflavin) from about 3 mg to 100 mg, Vitamin B6 (Pyridoxine) from about 3 mg to 100 mg, Vitamin C (ascorbic acid) in its various forms from about 25 mg to 1000 mg, zinc in its various bioavailable forms from about 1.67 mg to 75 mg, choline from about 3 mg to 100 mg, inositol from about 3.33 mg to 100 mg.

13. Another combination would be Vitamin E, in any of its various forms in the range of about 13 to 100 i.u., biotin in the range of about 3 mcg to 100 mcg, folic acid in the range of about 13 mcg to 100 mcg, niacin in any of its forms in the range of about 3 mg to 100 mg, pantothenic acid from about 3 mg to 100 mg, Vitamin B1 from about 3 mg to 100 mg, Vitamin B12 from about 3 mcg to 50 mcg, Vitamin B2 (riboflavin) from about 3 mg to 100 mg, Vitamin B6 (Pyridoxine) from about 3 mg to 100 mg, Vitamin C (ascorbic acid) in its various forms from about 25 mg to 1000 mg, zinc in its various bioavailable forms from about 1.67 mg to 75 mg, choline from about 3 mg to 100 mg, inositol from about 3.33 mg to 100 mg.

14. Another combination would be Vitamin E, in any of its various forms in the range of about 13 to 100 i.u., biotin in the range of about 3 mcg to 100 mcg, folic acid in the range of about 13 mcg to 100 mcg, niacin in any of its forms in the range of about 3 mg to 100 mg, PABA from about 3 mg to 100 mg, Vitamin B1 from about 3 mg to 100 mg, Vitamin B12 from about 3 mcg to 50 mcg, Vitamin B2 (riboflavin) from about 3 mg to 100 mg, Vitamin B6 (Pyridoxine) from about 3 mg to 100 mg, Vitamin C (ascorbic acid) in its various forms from 25 mg to 1000 mg, zinc in its various bioavailable forms from about 1.67 mg to 75 mg, choline from about 3 mg to 100 mg, inositol from about 3.33 mg to 100 mg.

15. Another combination would be 11, above without the B12.

16. Another combination would be 11, above without the B2.

17. Another combination would be 11, above without the B6.

18. Another combination would be 11, above without the choline.

19. Another combination would be 11, above without the choline or inositol.

20. Another combination would be 19 without the biotin.

21. Another combination would be 19 without the folic acid.

22. Another combination would be 19 without the niacin.

23. Another combination above would be 19 without the PABA.

24. Another combination would be 19 without the pantothenic acid.

25. Another combination would be 19 without the B12.

26. Another combination would be 19 without B2.

27. Another combination would be 19 without B6.

28. Another combination would be 19 without biotin or folic acid.

29. Another combination would be 28 without niacin.

30. Another combination would be 28 without PABA.

31. Another combination would be 28 without pantothenic acid.

32. Another combination would be 28 without B12.

33. Another combination would be 28 without B2.

34. Another combination would be 28 without B6.

35. As a further variation, alpha lipoic acid, about 50 to 100 mg, and/or selenium, about 200 to 400 mcg, can be included in any of the above formulations. Each of them has been found effective against certain types of infection which would also pertain to acne.

36. Any of the above formulations, further including oregano and parsley aquaresin as in the chart below.

It is important to note that the above variations reflect the fact that there are different causes of acne in different persons, and the different causes respond to some extent to different vitamin and mineral treatments. Many of the different individual components could be eliminated while still resulting in a composition that proves an effective treatment for much of the population of acne patients.

### Test Results

A total of 200 test candidates, ages 13 to 54, were tested with the formulation. They were asked to take two per day and to report whether there were any changes in their skin. As acne manifests itself differently, from mild to moderate to extensive, depending on the person and his condition at any given time, the candidates were simply asked to describe the change, if any was observed, in their skin. Two candidates with cystic acne were simultaneously treated with Accutane, which rendered their results confusing, at best. The Accutane-taking candidates had quite ruddy skin tones, looking like a rash both at the beginning and end of their trial. We do not therefore have an opinion as to whether the formulation is efficacious in the cystic acne population.

The test population was comprised of 200 people ages 13 to 54. Of all test candidates, 73.84% showed efficacy. Ages 13 to 17 had an efficacy rate of 74.6%, ages 18 and over an efficacy rate of 73.39%. Test subjects were not considered valid if they failed to take the product for the requisite time period. 26.16% of all test candidates reported no efficacy, 25.40% of 13 to 17 year olds and 26.61% of ages 18 and over. Observations included the description of preliminary results within a 12 hour period, manifesting as a reduction in the size of the acne break-outs and a general calming down of the outbreaks. Additional observations include the prevention of outbreaks proceeding through their normal course, from outset and rather, instead, being stopped.

Many test candidates have continued to ask for additional and/or ongoing supplies of product, a further indication of their positive finding.

On the following pages are preferred ranges for the formulation. The testing described above utilized the lowest amounts in the table, as the objective was to find the minimum amount necessary to achieve the desired result, and the test candidates took two per day, thus twice the minimum each day for each component.

### Brief Manufacturing Description

Manufacturing the product in a preferred gummy-candy embodiment will follow normal manufacturing process of gummy jelly candies, excluding sugar.

A solution of gelatin is prepared by soaking dried gelatin granules in water and heated at a temperature to sufficiently melt and solubilize the gelatin. A solution of sugar alcohols is heated to about 93% soluble solids and then cooled to below the boiling temperature of water. The solubilized gelatin is blended with the blend of vitamins and minerals until homogeneous. The gelatin solution is added to the sugar alcohol syrup and blended together along with flavors, colors and acidulants. The syrup solids at this point will be about 78-79% soluble solids. The blended syrup is deposited into starch mold impressions and allowed to solidify. The starch mold will draw excess moisture from the gelatin-based gel. Typical time to achieve desired level of moisture is 24 hours. The finished solids of the product will be about 82% soluble solids. The set candies are removed from the starch and the candies are lightly coated with fat and wax based film to keep the finished candies separate. The product is then transferred for packaging.

Ranges of Composition of Vitamin Blend Per 150 mg
(Target for 1 Unit)

| Component | Weight Range |
|---|---|
| Vitamin E (as acetate, USP-FCC) | 13.33 IU–100 IU |
| Biotin (FCC) | 3.33 mcg–100 mcg |
| Folic Acid (USP-FCC) | 13.33 mcg–100 mcg |
| Niacin (as Niacinamide, USP) | 3.33 mg–100 mg |
| PABA (USP) | 3.33 mg–100 mg |
| Pantothenic Acid (as D-Calcium | 3.33 mg–100 mg |
| Pantothenate, USP) | |
| Vitamin B1 (as Thiamin Mononitrate, USP-FCC) | 3.33 mg–100 mg |
| Vitamin B12 (as Cyanocobalamin, USP) | 3.33 mcg–50 mcg |
| Vitamin B2 (as Riboflavin, USP-FCC) | 3.33 mg–100 mg |
| Vitamin B6 (as Pyridoxine HCl, USP-FCC) | 3.33 mg–100 mg |
| Vitamin C (as Ascorbic Acid, USP-FCC) | 25 mg–1000 mg |
| Zinc (as Zinc Acetate) | 1.67 mg–75 mg |
| Choline (as Choline Bitartate, USP) | 3.33 mg–100 mg |
| Inositol (FCC) | 3.33 mg–100 mg |
| Maltodextrin (as Maltrin-100) | Quantity sufficient |
| (as a binding agent, not an active | to hold together |
| ingredient) | |
| Oregano & Parsley Aquaresin Solution | 0.01 g |

The invention further encompasses all edible forms of each component, when combined with the others, e.g. zinc as zinc sulfate rather than zinc acetate.

In another preferred form of the invention the above formulation further includes alpha-lipoic acid and selenium, in the ranges of about 10-100 mg, and about 400 mcg, respectively.

As noted above, gummy candies are one preferred embodiment, and the invention includes the incorporation of the formulation in other candy or confection, including candy bars, jelly beans, and other candy, preferably without sugar.

Some variations from the above table are possible while still gaining the principal benefits of the invention. Basically, the invention in broad terms encompasses a daily oral dose of a formulation that includes the combination of vitamin B (in a range of B vitamins), vitamin C, zinc, and vitamin E. A broad spectrum of the B vitamins, as listed in the above table, is preferred but is not absolutely necessary. Fewer than all of these B vitamins can be included in the formulation. The minimum quantities per day are approximately twice the amounts in the table, i.e. the table amount, taken twice per day.

Note that the above list of components and ranges includes four of the B vitamins (as well as pantothenic acid, which is sometimes identified as vitamin B5). It is preferred, but not absolutely necessary, that all or substantially all the B vitamins be included because, as explained above, the vitamin B complex components interact with each other. An excess of one or several of the B vitamins will diminish another, or several others. It should be understood that omission of several of the B vitamins, or even many of the B vitamins, may still provide an effective treatment against acne, but this may cause deficiencies in other respects, which can possible lead to other problems.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

### REFERENCES

1. Aamodt R L, Rumble W F, Johnston G S et al. Absorption of orally administrated 65Zn (zinc) by normal human subjects. Am J Clin Nutr 1981; 34: 2648-2652.
2. Abdel, K. M., El Mofty, A., Ismail, A., Bassili, F. Glucose tolerance in blood and skin of patients with acne vulgaris. Ind J Derm 1977; 22:139-149
3. \dot{A}gren M S. Studies on zinc in wound healing. Acta Derm Venereol Suppl 1990; 154:1-36 [review].
4, \dot{A}gren MS. Zinc in wound repair. Arch Dermatol 1999; 135:1273-4 [letter].
5. Allison J R. The relation of hydrochloric acid and vitamin B complex deficiency in certain skin diseases. South Med J 38: 235-241, 1945. Bendich A, Cohen M. Nutrition and Immunology. New York: Alan R. Liss. p 101-123, 1988.
6. Alvarez O M, Gilbreath R L. Effect of dietary thiamine on intermolecular collagen cross-linking during wound repair: a mechanical and biochemical assessment. J Trauma 1982;22:20-4.
7. Anderson R, Oosthuizen R, Maritz R et al. Effects of increasing weekly doses of ascorbate on certain cellular and humoral immune functions in normal volunteers. Am J Clin Nutr 33: 71-76, 1980.
8. Aprahamian M, Dentinger A, Stock-Damge C, et al. Effects of supplemental pantothenic acid on wound healing: experimental study in rabbit. Am J Clin Nutr 1985; 41:578-89.
9. Ayres S Jr., Mihan R. Acne vulgaris: therapy directed at pathophysiologic defects. Cutis 1981; 28 (1):41-42
10. Barr W, Pyridoxine Supplements in the Premenstrual Syndrome, Practitioner 228: 425-7, 1984.
11. Bassler K H. Megavitamin therapy with pyridoxine. Int J Vit Nutr Res 58: 105-118, 1988.
12. Berman M K, et al. Vitamin B6 in Premenstrual Syndrom, J Am Diet Assoc 90: 859-61, 1990.
13. Block M T. Vitamin E in the treatment of diseases of the skin. Clin Med January pp. 31-4, 1953.
14. Bosse M D, Axelrod A E. Wound healing in rats with biotin, pyridoxin, or riboflavin deficiencies. Proc Soc Exp Biol Med 1948; 67:418-21.
15. Boelsma, Esther, Henk F J Hendriks, and Len Roza. Nutritional skin care: health effects of micronutrients and fatty acids Am. J. Clinical Nutrition, May 2001; 73: 853-864.
16. Cathcart, R F. Vitamin C as a detoxifying agent. Presented at the annual meeting of the Orthomolecular Medical Society, San Francisco, Jan. 21, 1978.
17. Cathcart, R F. Vitamin C—The missing stress hormone. Presented at the annual meeting of the Orthomolecular Medical Society, San Francisco, Mar. 3, 1979.
18. Cheraskin E, Ringsdorf W M Jr, Medford F H et al. The ‘ideal’ daily vitamin B, intake. J Oral Med 33: 77-79, 1978.
19. Cheraskin E, Ringsdorf W M Jr. How much refined carbohydrate should we eat? Am Lab 6: 31-35, 1974.
20. Chichester, U K: John Wiley & Sons, Ltd. All rights reserved. Vitamin supplementation for preventing miscarriage (Cochrane Review) Rumbold A, Middleton P, Crowther
21. DiMascio P, Murphy M, Sies H. Antioxidant and defense systems. The role of carotenoids, tocopherols and thiols. Am J Clin Nutr 53: S194-200, 1991.
22. Dreno B, Amblard P, Agache P, et al. Low doses of zinc gluconate for inflammatory acne. Acta Dermatovener (Stockholm) 1989; 69:541-3.
23. Engel E D et al. Diabetes melitus: Impaired Wound Healing from Zinc Deficiency. J Am Pod Assoc 71: 536-44, 1981.
24. Evans, William J. Vitamin E, vitamin C, and exercise Am. J. Clinical Nutrition, August 2000; 72:647-652.
25. Fink M, Fink J. Treatment of herpes simplex by alpha-tocopherol (Vitamin E). Br Dent J 148:246, 1980.
26. Finnerty E F. Topical zinc in the treatment of herpes simplex. Cutis 37; (2):130-1, 1986.
27. Fitzherbert J. Genital herpes and zinc. Med J Aust 1:399, 1979.
28. Folkers K, Morita M, McRee Jr. J. The activities of coenzyme Q10 and vitamin B6 for immune response. Biochem Biophys Res Commun 193:88-92, 1993.
29. Gordon Y J et al. Irreversible inhibition of herpes simplex virus replication in BSC-1 cells by zinc ions. Antimicrob Agents Chemother 8; (3):377-80, 1975.
30. Gupta A K, Mital H. S. Cyanocobalamin (vitamin B12) in the management of Herpes Zoster. Indian Pract 20; (7):457-9, 1967.
31. Hansen, Marianne, Samir Samman, Lene T Madsen, Mikael Jensen, Sven S Sorensen, and Brittmarie Sandström. Folic acid enrichment of bread does not appear to affect zinc absorption in young women. Am. J. Clinical Nutrition, July 2001; 74: 125-12
32. Hegazi S M et al. Effect of Zinc Supplementation on Serum Glucose, Insulin, Glucagon, Glucose-6-Phosphatase and Mineral Levels in Diabetics. J Clin Biochem Nutr 12: 209-15, 1992.
33. Herbert, V. Vitamin B-12. In: Present knowledge in nutrition. 7th edn. Washington D.C.: International Life Sciences Press. 1996: p 191-205.
34. Heyblon R. Vitamin B12 in herpes zoster. Abstract. JAMA 146:1338, 1951.
35. Hillström, L Pettersson L, Hellbe L, et al. Comparison of oral treatment with zinc sulfate and placebo in acne vulgaris. Br J Dermatol 1977; 97:681-4.
36. Joliffe N, Rosenblum L A, Sawhill J. Effects of pyridoxine (vit B6) on resistant adolescent acne. J Invest Dermatol 1942; 5:143-8
37. Juhlin L et al. Blood glutathione-peroxidase levels in skin diseases: Effect of selenium and vitamin E treatment. Acta Derm Venereal (Stockh) 62; 3: 211-4, 1982.
38. Kondo T, Toda Y, Matsui H. Effects of exercise and sleep deprivation on serum zinc. J Trace Elem Exp Med 1990; 3: 324-354, 1990. Lansdown A B G. Zinc in the healing wound. Lancet 1996; 347:706-7 [editorial].
39. Scott W Leonard, Carolyn K Good, Eric T Gugger, and Maret G Traber. Vitamin E bioavailability from fortified breakfast cereal is greater than that from encapsulated supplements Am. J. Clinical Nutrition, January 2004; 79:86-92.
40. Leung L H. Pantothenic acid deficiency as the pathogenesis of acne vulgaris. Med Hypotheses. 1995; 44(6):490-2
41. Liszewski R F. The effect of zinc on wound healing: a collective review. J Am Osteopath Assoc 1981; 81:104-6 [review].
42. Mazzotta M Y. Nutrition and wound healing. J Am Podiatr Med Assoc 1994; 84:456-62 [review].
43. Michaelsson G. Oral zinc in acne. Acta Dermatovener (Stockholm) 1980; Suppl 89:87-93 [review].
44. Michaelsson G, Edqvist L E. Erythrocyte glutathione peroxidase activity in acne vulgaris and the effect of selenium and vitamin E treatment. Acta Derm Venereol. 1984; 64(1):9-14
45. Nead D E. Effective vitamin E treatment for ulcerative herpetic lesions. Dent Survey 52; (7):50-1, 1976.
46. Pascoe G A, Fariss M W, Olafsdottir K et al. A role of vitamin E in protection against cell injury. Maintenance of intracellular glutathione precursors and biosynthesis. Eur J Biochem 166: 241-247, 1987.
47. Palmieri B, Gozzi G, Palmieri G. Vitamin E added silicone gel sheets for treatment of hypertrophic scars and keloids. Int J Dermatol 1995; 34:506-9
48. Pauling, L. Vitamin C and the Common Cold. W.H. Freeman and Company, San Francisco, 1970.
49. Pocoit F, Reimers J I and Andersen H U. Nicotinamide: Biological Actions and Therapeutic Potential in Diabetes Prevention. Diabetologia 36: 574-76, 1993.
50. Pories W J, Henzel J H, Rob C G, Strain W H. Acceleration of healing with zinc sulfate. Ann Surg 1967; 165:432-6.
51. Posacki C, et al. Plasma, Copper, Zinc, and Magnesium Levels in Patients with Premenstrual Tension Syndrome, Acta syndrome: Obstet Gynecol Scand 73; 452-5, 1994.
52. Pozzilli P et al. The Potential Role of Nicotinamide in the Secondary Prevention of IDDM. Diabetes Metabol Rev 9: 219-30, 1993.
53. Prasad J S. Effect of vitamin E supplementation on leukocyte function. Am J Clin Nutr 33: 606-608, 1980.
54. Raqib, Rubhana, Swapan Kumar Roy, Muhammad Jubayer Rahman, Tasnim Azim, Syeda Shegufta Ameer, Jobayer Chisti, and Jan Andersson. Effect of zinc supplementation on immune and inflammatory responses in pediatric patients with shigellosis Am. J. Clinical Nutrition, March 2004; 79: 444-450.
55. Richardson L R, Brock R. Studies of reproduction in rats using large doses of vitamin B12 and highly purified soybean proteins. J Nutr 1956; 58: 135-145.
56. Ringsdorf W M Jr, Cheraskin E. Vitamin C and human wound healing. Oral Surg 53: 231-236, 1982.
57. Roberts P et al. Vitamin C and inflammation. Med Biol 62:88, 1984.
58. Said, Hamid M, Biotin: the forgotten vitamin Am. J. Clinical Nutrition, February 2002; 75:179-180.
59. Sandstead H H. Understanding zinc: Recent observations and interpretations. J Lab Clin Med 1994; 124:322-7
60. Snider B, Dietman D F. Pyridoxine therapy for premenstrual acne flare. Arch Dermatol 1974; 110:130-1 [letter].
61. Steinberg C L. Vitamin E and collagen in the rheumatic diseases. Ann N Y Acad Sci 52:380-9, 1949.
62. Solomon L R et al. Erythrocyte O2 Transport and Metabolism and Effects of Vitamin B6 Therapy in Type II Diabetes mellitus. Diabetes 38: 881-86, 1989.
63. Stone S. Pyridoxine and thiamine therapy in disorders of the nervous system. Dis Nerv Sys 11; 5:131-8, 1950.
64. Stone, I. The Healing Factor: Vitamin C Against Disease. Grosset and Dunlap, New York, 1972.
65. Takihara H, Cosentino M J, Cockett A T. Effect of low-dose androgen and zinc sulfate on sperm motility and seminal zinc levels in infertile men. Urology 22: 160, 1983.
66. Tenaud I, Sainte-Marie I, Jumbou O, et al. In vitro modulation of keratinocyte wound healing integrins by zinc, copper and manganese. Br J Dermatol 1999; 140:26-34.
67. Vaxman F, Olender S, Lambert A, et al. Can the wound healing process be improved by vitamin supplementation? Experimental study on humans. Eur Surg Res 1996; 28:306-14.
68. Vaxman F, Olender S, Lambert A, et al. Effect of pantothenic acid and ascorbic acid supplementation on human skin wound healing process. Eur Surg Res 1995; 27:158-66.
69. Verma K C, Saini A S, Dhamija S K. Oral zinc sulphate therapy in acne vulgaris: a double-blind trial. Acta Dermatovener (Stockholm) 1980; 60:337-40.
70. Wagner P A, Bailey L B, Christakis G J, Dinning J S. Serum zinc concentrations in adolescents as related to sexual maturation. Human Nutr. Clin Nutr 39C: 459, 1985.
71. Welsh A L. Lupus erythematosus: Treatment by combined use of massive amounts of pantothenic acid and vitamin E. Arch Dermatol Syphilol 70: 181-98, 1954.
72. Balch, J. F., Balch, P. A., Prescription for Nutritional Healing, Avery Publishing Group Inc., New York, USA, 1990.
73. Wyngaarden, J. B., Smith, L. H., Cecil Textbook of Medicine, 17th edn., W. B. Saunders Company, Philadelphia, USA, 1985.
74. Kirschmann, G. J., Kirschmann, J. D., Nutrition Almanac, 4th edn, McGraw Hill, New York, USA, 1996.
75. Davis, A., Let's Get Well, George Allen and Unwin Ltd., London, 1978.
76. Holman, P., Journal of the Australasian College of Nutritional & Environmental Medicine, Vol. 14, No. 1, July 1995, pages 5-16.

We claim:

1. A method for treating acne, comprising:
taking orally a daily dosage of at least the following vitamins and minerals: a range of B vitamins, including vitamin B1, at least about 7 mg, vitamin B12, at least about 7 mcg, vitamin B2, at least about 7 mg and vitamin B6, at least about 7 mg; vitamin C, at least about 50 mg; vitamin E, at least about 27 IU; and zinc, at least about 3 mg.

2. The method of claim 1, wherein the vitamins and minerals are taken as a combined formulation, in amounts half of those listed in claim 1, taken twice daily.

3. The method of claim 1, wherein the vitamins and minerals are taken as a combined formulation in the form of a gummy candy.

4. The method of claim 3, wherein the gummy candy formulation is without sugar.

5. The method of claim 1, wherein the vitamins and minerals further include pantothenic acid, at least about 7 mg.

6. The method of claim 1, wherein the vitamins and minerals include choline, at least about 7 mg.

7. The method of claim 1, wherein the vitamins and minerals include inositol, at least about 7 mg.

8. The method of claim 1, wherein the vitamins and minerals include biotin, at least about 7 mcg.

9. The method of claim 1, wherein the vitamins and minerals include folic acid, at least about 27 mcg.

10. The method of claim 1, wherein the vitamins and minerals include niacin, at least about 7 mg.

11. The method of claim 1, wherein the vitamins and minerals include PABA, at least about 7 mg.

12. The method of claim 1, wherein the range of B vitamins includes all B vitamins.

13. The method of claim 1, wherein the range of B vitamins includes substantially all B vitamins.

14. The method of claim 1, wherein the vitamins and minerals are taken in a combined formulation in the form of a gummy candy, and the formulation including maltodextrin.

15. The method of claim 14, wherein the formulation is without sugar.

16. The method of claim 1, wherein the vitamins and minerals are taken in a combined formulation that further includes oregano and parsley aquaresin solution, at least about 10 mg.

17. The method of claim 1, wherein the vitamins and minerals include alpha lipoic acid, at least about 50 mg.

18. The method of claim 1, wherein the vitamins and minerals include selenium, at least about 200 mcg.

19. A formulation for the treatment and prevention of acne, comprising:
a gummy candy substrate, without sugar, and
vitamins and minerals contained in the gummy candy substrate, including at least the following: a range of substantially all B vitamins each in an effective amount, in combination with remaining components listed herein, to treat acne with two doses of the formulation taken orally daily; vitamin E, at least about 13.33 IU; biotin, at least about 3.33 mcg; folic acid, at least about 13.33 mcg; niacin, at least about 3.33 mg; PABA, at least about 3.33 mg; pantothenic acid, at least about 3.33 mg; vitamin C, at least about 25 mg; zinc, at least about 1.67 mg; choline, at least about 3.33 mg; and inositol, at least about 3.33 mg.

20. An edible formulation for the treatment and prevention of acne, comprising:
a sugar-free candy substrate, and
vitamins and minerals contained in the candy substrate, including at least the following: a spectrum of substantially all B vitamins, vitamin E, biotin, folic acid, niacin, PABA, pantothenic acid, vitamin C, and zinc, all in effective amounts in combination to treat acne with two doses of the formulation taken orally daily.

21. The method of claim 1, wherein the vitamins and minerals include inositol, biotin, folic acid, niacin and PABA.

22. The method of claim 1, wherein the vitamins and minerals include at least some of the following: inositol, biotin, folic acid, niacin and PABA.

23. The method of claim 1, wherein the vitamins and minerals include at least some of the following: inositol, at least about 7 mg; biotin, at least about 7 mcg; folic acid, at least about 27 mcg; niacin, at least about 7 mg; and PABA, at least about 7 mg.

24. The method of claim 23, wherein the vitamins and minerals include selenium, at least about 200 mcg, and wherein the vitamins and minerals are taken in a combined formulation that further includes oregano and parsley aquaresin solution, at least about 10 mg.
